(19) United States
(12) Patent Application Publication
(10) Pub. No.: US 2005/0015114 A1
(43) Pub. Date: Jan. 20, 2005

(54) Piercing article and intermediate product for manufacturing such a piercing article

(76) Inventor: Eugen Seibold, Salzweg (DE)

Correspondence Address: FITCH EVEN TABIN AND FLANNERY, 120 SOUTH LA SALLE STREET, SUITE 1600, CHICAGO, IL, 60603-3406, US

(21) Appl. No.: 10/704,323

(22) Filed: Nov. 7, 2003

Related U.S. Application Data
(63) Continuation of application No. PCT/EP02/04716, filed on Apr. 29, 2002.

(30) Foreign Application Priority Data
May 7, 2001 (DE) 20107674.8

(51) Int. Cl. A61B 17/08

(52) U.S. Cl. 606/219

(57) ABSTRACT

The invention relates to a piercing article comprising a longitudinal insertion member in the form of a pin. The inventive piercing article is mainly composed of an alkylene-fluoroalkylene polymer material, especially an ethylene-tetrafluoroethylene (ETFE) material. The invention further relates to an intermediary product for the manufacturing of said piercing article. Said intermediary product comprises an elongated member in the form of a wire or rod made of an alkylene-fluoroalkylene copolymer material, especially an EFTE material.

The present invention relates to a piercing article and an intermediary product for manufacturing such a piercing article. Further, the invention relates to the use of a special material for the manufacture of a piercing article or an intermediary product for the manufacturing of a piercing article.

A piercing article is to be understood as an object used to adorn the body and as jewelry for intimate piercing, which, similar to an ear stud, is pushed by means of piercing through a wound tract that has either already healed or is fresh and was made in a part of the human body, and which is removably attached. The mounting of the piercing article takes place with special devices similar to surgical instruments. The piercing article is thoroughly sterilized before the operation, and especially when to be used for the first time.

A piercing article comprises as a rule a longitudinal insertion member in the shape of a pin, which forms an essential part of the piercing article pushed through the wound tract. The pin-shaped insertion member can be shaped to be straight, curved, bent to be annular or even in the shape of a tube. The insertion members of most piercing articles comprise at one end an insertion member head, the diameter of which is greater than that of the insertion member. The insertion member head forms a counterpiece at one corporal side of the wound tract, which prevents the piercing article from slipping out of the wound tract. At the other, extra-corporal wound-tract side, the piercing article is provided usually with a locking member which is releasably connected with a free end of the insertion member projecting out of the wound tract. The piercing article is connected in this manner with the pierced human body part such that it cannot be lost. Most of the known piercing articles have insertion members, insertion member heads and locking members manufactured of titanium or a stainless steel, i.e. what is called “surgical steel”. However, insertion members are also used which are produced from polytetrafluoroethylene (PTFE).

Conventional piercing articles of the type described above are known, for example, from the production catalogues for 1998 of SIN-A-MATIC of 90762 Fürth, Germany, Cold Steel of London, NW1 8QS, England and The Wildcat Collection of Brighton, BN1 2HN, England.

The previously known piercing articles, however, have various disadvantages. For example, the stainless steels used contain, even if only in minor amounts, nickel which makes them unsuitable for piercing articles to be made use of for the first time. Further, when using titanium and steel materials, conglutination can easily occur despite the high quality of the surface of the piercing article due to wound secretion which causes inadequate healing and an impairment of the well-being of the wearer of the piercing article, irritation of the wound as well as a greater risk of infection. It has been shown, moreover, that as a consequence of said metal materials, disagreeable irritation to tissue and/or allergic reactions can occur, especially at the insertion member head located at a corporal side of the wound tract which lies, in comparison to the actual insertion member, with a relatively large contact surface on the adjacent human tissue. What are called bottom-lip piercings, in which the insertion member head rests between a labial area of the oral cavity and the adjacent gum on the tissue, have even given rise to complications such that said metal material has caused, after a longer period of use, a partial atrophy of the gums in the area of contact.

PTFE piercing articles, in contrast thereto, are suited for first time use. Also, the anti-adhesive properties of PTFE surfaces are better than are metal materials as regards possible conglutination due to wound secretion. PTFE piercing articles, however, can only be manufactured by a cutting process. The quality of the surface attainable thereby is considerably worse than metal piercing articles since furrows or grooves are left from the processing. These furrows or grooves can result in a kind of positive connection with hardening wound secretion so that, despite the good anti-adhesive properties per se, there is on the other hand a disadvantageous effect comparable to wound secretion conglutination. Also, the entire piercing article surface is relatively coarse or even shape-edged as a whole due to the surface structure caused by the material and processing, which can result in irritation to the tissue, bad healing and even injuries. Moreover, a PTFE piercing article has only properties mechanically unsatisfactory which are specially disadvantages in insertion members with a lesser diameter. For example, during attachment of the locking member which is usually screwed onto the free end of the insertion member, the insertion member is slightly twisted or is even greatly distorted such that it is suitable at the most for only one single application. Furthermore, the worse mechanical properties of PTFE also place limits as regards the possibility to process using a cutting process of piercing articles with especially small diameters. PTFE materials suited for piercing articles are also obtainable in an amount of commercial interest only in a uniform white color which contrasts greatly with the color of human tissue and is found by many users to be objectionable.

The invention, therefore, is based on the technical problem to create a piercing article which avoids as far as possible the disadvantages of the prior art, but which can still be manufactured as simply and effectively as possible. A further technical problem of the invention is to provide a suitable intermediary product for the manufacturing of such a piercing article. A further technical problem of the invention is the use of an especially suitable material for the manufacture of a piercing article or an intermediary product for the manufacturing of a piercing article.

The first-mentioned technical problem is solved by a piercing article having the features of claim 1.

This piercing article having a longitudinal pin-shaped insertion member is produced essentially completely of an alkylene-fluoralkylene copolymer material.

Within the meaning of the present invention, non-fluorinated olefins are to be understood by alkylene, such as, for example, ethylene or propylene. Fluoroalkylene can, for example, be tetrafluoroethylene, vinyl fluoride, vinylidenfluoride or the like.

“Essentially” means here that at least those areas coming into contact with the human tissue of the user of the piercing article consist of alkylene-fluoroalkylene copolymer material. It is also possible within the meaning of the invention that metallic components are embedded within the alkylene-fluoroalkylene copolymer material of the piercing article or its insertion member such that there can be no direct contact between these components and the human tissue. However, the invention includes in particular a piercing article which (except for any extra-corporal ornament parts possibly provided) consists completely, i.e. without further restrictions, of an alkylene-fluoroalkylene copolymer material. The pin-shaped insertion member of the piercing article can be designed not only to be straight, but also curved, annular, spiral-shaped, tube-shaped or in any other suitable manner. The insertion member preferably has a circular cross-section, with, however, the invention not being restricted to this cross-section form. The dimensions, in particular the length and diameter of the piercing article, can vary depending on the form of application. Diameters of approximately 0.8 to 10 mm are considered as common for piercing articles, however, these figures do not represent a restriction. The piercing article according to the invention can, for example, be manufactured by a resin injection moulding process in which a suitable alkylene-fluoroalkylene copolymer starting material is extruded and injected into a form. Moreover, the piercing article can also be produced by means of an intermediary product according to claim 11, which will be described in more detail below.

The piercing article according to the invention of an alkylene-fluoroalkylene copolymer material has an excellent pharmaceutical compatibility and is thus especially suited also to be made use of for the first time. Because its high surface quality is obtainable as opposed to conventional PTFE piecing articles as well as due to the anti-adhesive properties of the alkylene-fluoroalkylene copolymer material, conglutination due to wound secretion can be effectively avoided, as occurs with known metal and PTFE piercing articles. Moreover, it has been found that fresh wound tracts which receive piercing articles according to the invention heal better and more quickly. The risk of irritation to the wound or tissue or an infection is considerably reduced with the piercing article according to the invention and the wearing comfort is improved for the user. Allergic reactions are not as of yet known with piercing articles according to the invention. The alkylene-fluoroalkylene copolymer material is to be considered more as having an anti-allergic effect. In particular, the danger of unpleasant irritation to tissue and/or allergic reactions is thus minimized at those areas of a piercing article having contact over a larger surface with adjacent human tissue. For example, with bottom-lip piercings where a large surface of the piercing article according to the invention rests between a labial area of the oral cavity and the adjacent gums on the tissue, the risk of a partial atrophy of the gums is almost completely eliminated, in contrast to the known prior art.

The alkylene-fluoroalkylene copolymer material of the piercing article created by the invention can be provided, as a standard measure, in a transparent color or, however, can be dyed in a simple, effective and inexpensive manner such that the cosmetic needs of a wearer can be easily taken into account. The alkylene-fluoroalkylene-copolymer-material piercing article of the present invention also has as regards purely physical and/or manufacturing aspects not inconsiderable advantages over conventional piercing articles. As such the alkylene-fluoroalkylene copolymer material has, as opposed to PTFE, better mechanical characteristic values which make it possible to manufacture by cutting even piercing articles with especially small insertion-member diameters. Reference is made here to the fact that the manufacture by chip-producing means as mentioned above is fundamentally possible with good results, however, is usually not necessary since the alkylene-fluoroalkylene copolymer material can be excellently processed, as opposed to PTFE, by using injection-molding technology. A further finishing process using cutting means is thus, as a rule, not necessary, and the injected alkylene-fluoroalkylene-copolymer-material piercing article already has, even after the injection molding step, an excellent surface quality. A piercing article according to the invention and formed in this manner without cutting is therefore preferred. Due to the better mechanical properties and a greater degree of stability, the insertion members of a piercing article according to the invention will not be so easily liable to plastic deformation by torsion forces or even twisting off if the insertion member only has a small diameter, and a locking member is screwed onto the free end of the insertion member. The locking member can, to the contrary, be repeatedly attached and removed without the piercing article experiencing thereby structural failure. It is, therefore, fundamentally suited for multiple applications. In addition thereto, the alkylene-fluoroalkylene copolymer material also has a good flexural rigidity which is also to be considered as positive.

An ethylene tetrafluoroethylene (ETFE) material has proven to be especially suited as a preferred alkylene-fluoroalkylene copolymer material for piercing articles of the said kind.

Further preferred embodiments of the piercing article according to the invention are the subject matter of the sub-claims 2 through 10.

The second technical problem underlying the invention is solved by an intermediary product with the features of claim 11.

This intermediary product for manufacturing a piercing article according to claim 1 comprises an elongated member in the form of a wire or rod made of alkylene-fluoroalkylene copolymer material. The elongated member in the form of a wire or rod can be provided in predetermined, defined lengths or, however, in a continuous form. The member in the form of a wire or rod can be straight, curved or, for example, in the form of a roll. The cross-section shape of the wire- or rod-shaped member is preferably circular, however, it can, depending on the form of application, also have any other suitable form desired. It is not absolutely necessary that the member in the form of a wire or rod always has the same cross-section form and/or dimension throughout its length.

The intermediary product according to the invention also offers the advantages explained in detail above in connection with the piercing article according to the invention. Moreover, the intermediary product according to the invention allows, along with the possibility of the resin injection moulding process mentioned above, an especially simple and effective manner of manufacturing a piercing article according to the invention. Thus, for example, the longitudinal member in the form of a wire or rod of the intermediary product can, for example, be deformed under the impact of heat, and the piercing article can by this means be shaped with its pin-shaped insertion member as well as other piercing-article areas without a special and expensive injection molding machine being necessary therefor. When the member in the form of a wire or rod of the intermediary product is correspondingly adapted to the dimensions of the piercing article, the steps necessary for manufacturing the piercing article can be considerably reduced. It can even be sufficient for some variations to simply cut the intermediary product to a length in order to obtain a suitable piercing article, for example, for the navel area of a human body. Which methods of manufacturing will be preferred, depends on the specific case.

Preferred embodiments of the intermediary product according to the invention are the subject matter of the sub-claims 12 through 14.

The third technical problem underlying the invention is solved by the use according to the invention of an alkylene-fluoroalkylene copolymer material for the manufacture of a piercing article or an intermediary product for manufacturing a piercing article, corresponding to the features of claim 15.

Preferred embodiments of the invention with additional embodiments details and further advantages are described and explained in detail below with reference to the enclosed drawings.

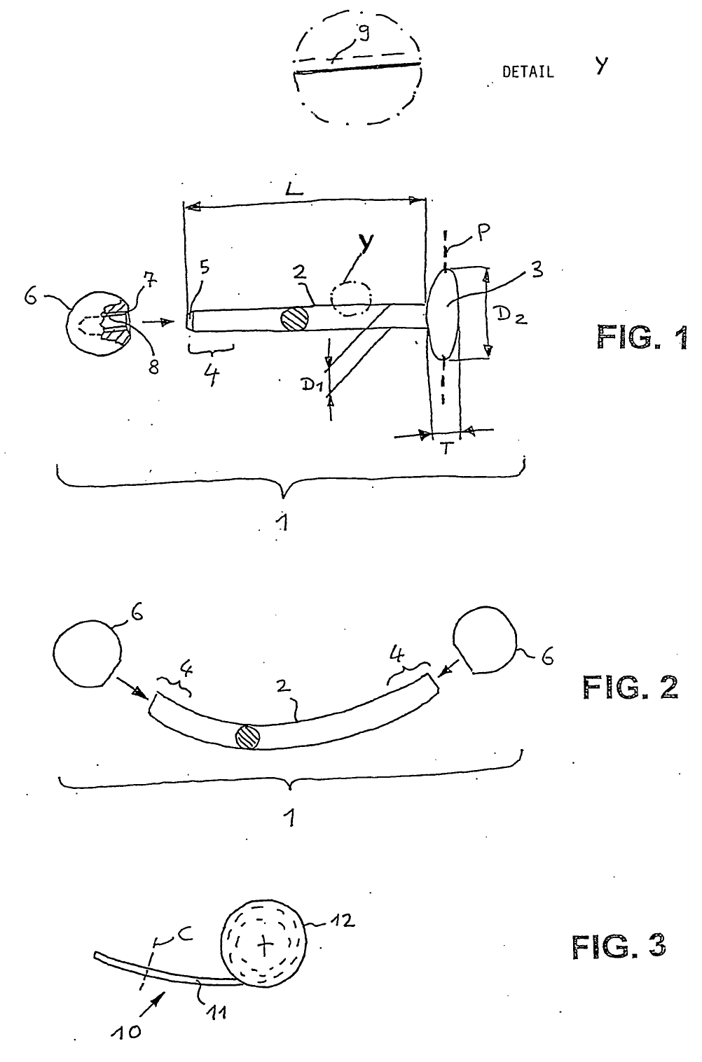

FIG. 1 is a schematic side view of a piercing article according to the invention according to a first embodiment;

FIG. 2 is a schematic side view of a piercing article according to the invention according to a second embodiment; and

FIG. 3 is a schematic side view of an intermediary product according to the invention provided in roll form, from which the piercing article according to FIG. 2 is manufactured.

In the following description and in the figures, the same components and parts will be characterized, to avoid repetition, also with the same reference number, if no differentiation is necessary.

In FIG. 1, a first embodiment of a piercing article 1 according to the invention is shown in a schematic side view, which can be used inter alia for what are called lower-lip piercings. This piercing article 1 consists in the present example completely of an alkylene-fluoroalkylene copolymer material. In the present case, an ethylene tetrafluoroethylene (ETFE) material is used as alkylene-fluoroalkylene copolymer material. The most important physical properties of the ETFE material of the piercing article 1 are revealed in the following table.

Important Physical Properties of the
ETFE Material Used

| | |
|---|---|
| Density [g/cm³] | 1.71-1.78 |
| Upper continued use temperature [° C.] | 150-180 |
| Combustibility [-] | self- |
| | extinguishing |
| Water absorption [%] | <0.1 |
| Breaking strength at 23° C. [N/mm²] | 36-48 |
| Breaking strength at 150° C. [N/mm²] | 8-12 |
| Elastic limit at 23° C. [N/mm²] | 24 |
| Elongation at break at 23° C. [%] | 200-500 |
| Modulus of elasticity in tension at | 500-1200 |
| 23° C. [N/mm²] | |
| Limit of bending stress at 23° C. | 25-30 |
| [N/mm²] | |
| Modulus of elasticity in bending | 1000-1500 |
| [N/mm²] | |
| Indentation hardness 132/60 [-] | 34-40 |
| Rockwell hardness R [-] | 45-55 |
| Shore hardness D [-] | 63-75 |
| Friction coefficient, dynamic against | 0.3-0.5 |
| steel. dry, [-] | |
| Melting temperature [° C.] | 265-275 |
| Linear heat-expansion coefficient | 8-12 |
| Heat conductibility at 23° C. [W/Kg*m] | 0.23 |

An ETFE material is used within the scope of the embodiment having the trade name TEFCEL®, which has the above-described properties. The invention is, however, restricted neither to this special trade product nor to an ETFE material as is given in the table. The ETFE material is furthermore dyed with a pharmaceutically compatible and/or food-safe dye in order to attain a color adapted to human tissue.

The piercing article 1 has a straight, longitudinal, pin-shaped insertion member 2 having a uniform circular cross-section. The length L of the insertion member 2 amounts to approximately 26 mm and its diameter D1 to approximately 1.6 mm. The insertion member 2 has at one end a disc-, spheri-cal-, or lens-shaped insertion member head 3 formed integrally, the diameter D2 of which is approximately 5 mm and thus greater than that of the insertion member 2. The thickness T of the insertion member head 3 amounts to approximately 1 mm. The insertion member head 3 consists, due to its structure integral with the insertion member 2, also of ETFE. The other, i.e. the free, end 4 of the insertion member 2 has a bevel 5 and thus an end area shaped in a tapered manner.

The piercing article 1 comprises moreover a locking member 6 connectable to the free end 4 of the insertion member 2. This locking member 6 is in the present case a sphere 6 consisting of a pharmaceutically compatible metal material and having a leveled sphere section 7 in which a threaded bore 8 is provided adjusted to the diameter of the insertion member 2. The threaded bore 8 functions as a connecting means, with which the spherical-like locking member 6 is connectable with the free end 4 of the insertion member 2. For this purpose, the sphere 6 is simply screwed extra-corporally onto the beveled free end 4 of the insertion member 2 after the piercing article 1 has been pushed through a fresh or already healed wound tract (e.g. in the musculus orbicularis oris or musculus depressor labii inferioris). Due to the combination of materials, the threading is self-tapping) or deforms the ETFE material so sufficiently that a solid connection, which can again be released, is produced and the piercing article 1 is fixed to the pierced body part in a manner so that it cannot get lost. The bevelling 5 makes the screwing-in easier, it is, however, not necessary. Since the entire length of the piercing article 1 is adjusted as a rule individually to the size of a user, it is usually sufficient to shorten the free end 4 simply with a cutting tool and, optionally, to remove any shape edges then given.

The locking member 6 can, fundamentally, take on any suitable form and is generally designed as an ornament. Instead of being of a metal material, the locking member 6 can also consist in part or in full of an alkylene-fluoroalkylene copolymer material, in particular of ETFE, or of another material, such as for example, of another suitable synthetic material or of wood. Depending on the material of the locking member 6, the free end 4 of the insertion member 2 is to be, where necessary, correspondingly adjusted to the connecting means of the locking member 6. It can, for example, be necessary with a locking member of alkylene-fluoroalkylene copolymer material, in particular an ETFE-locking member, to attach at the free end 4 an outer threading corresponding with the threaded bore of the locking member 6.

The areas of the ETFE piercing article 1 according to the invention which come into contact with the human tissue of the user are coated in the embodiment according to FIG. 1 with an anti-inflammatory agent 9, as is indicated in the detail Y by a thin dotted line. Instead of applying a coating, the ETFE material could naturally also be mixed as early as in the crude state or during the injection molding with an anti-inflammatory agent.

The piercing article 1 according to the invention is produced according to FIG. 1 in an injection molding process in a two-part injection mold, with the partitioning level P of the form, as indicated by a thick dotted line, extending transversly through the insertion member head 3.

FIG. 2 shows in a schematic side view a piercing article 1 according to the invention according to a second embodiment, and FIG. 3 shows a schematic side view of an intermediary product 10 according to the invention provided in the form of a roll, from which the piercing article of FIG. 2 is manufactured. It must be taken into consideration that FIGS. 2 and 3 are shown on different scales. The piercing article 1 of FIG. 2, for example, is used for what is called navel piercings.

As can be seen from FIG. 3, the intermediary product 10 comprises an elongated member 11 in the form of a wire or rod made of an alkylene-fluoroalkylene copolymer material, i.e. in the present example an ETFE material. This rod-shaped member 11 was manufactured by an extrusion method. The member 11 in the form of a wire or rod, a long length of which is wound onto a roll 12, has in the present example a circular cross-section. The ETFE material is the same as in the piercing article 1 of FIG. 1. The ETFE material is dyed with a pharmaceutical compatible and/or food-safe dye. When desired, the ETFE material can also be provided with an anti-inflammatory agent.

To manufacture the actual piercing article 1, the member 12 in the form of a wire or rod of the intermediary product 11 is cut with a separating tool to the length required, as is indicated in FIG. 3 by the reference C. By this means, a longitudinal insertion member 2 in the form of a pin is formed, as is shown in FIG. 2. Any possible sharp edges at the free ends 4, 4 of the insertion member 2 created by the cut C are removed.

The insertion member 2 is then pushed through with a suitable instrument the wound tract already healed or fresh so that the two free ends 4, 4 of the insertion member 2 project at both sides from the wound tract. Two locking members 6, 6, which correspond as regards their construction to the locking member 6 of FIG. 1, are each screwed to one free end 4, 4 of the insertion member 2 and thus the entire piercing article 1 is secured to the pieced body part in a manner so as to not to be lost.

Although in the embodiment according to FIG. 3 the intermediary product 10 was processed only with a separating tool, it is generally possible to process the intermediary product 10 in another way, for example, by thermoplastic deformation of a partial region of the intermediary product 10 to form an integral insertion member head, to alter the insertion member form and/or the insertion member diameter and the like.

The invention is not restricted to the above examples which are only to generally explain the basic idea of the invention. The piercing article according to the invention and the intermediary product according to the invention can, to the contrary, also assume within the scope of protection different embodiments than those described above. The piercing article and the intermediary product can comprise here in particular features which represent a combination of the respective individual features of the respective claims, with it, however, not being obligatory that all of these features are used in one embodiment. It is self-evident that in particular said dimensions of the piercing article can vary considerably depending on their use.

The reference numerals and letters in the claims, description and drawings are only for a better understanding of the invention and are not to restrict the scope of protection.

### List of Reference Numbers/Letters

These indicate:
1 Piercing article
2 Insertion member
3 Insertion member head
4 Free end of 2
5 Bevel
6 Locking member
7 Leveled spherical section of 6
8 Threaded bore in 6 and/or 7
9 Anti-inflammatory agent
10 Intermediary product
11 Member of 11 in the form of a wire or rod
12 Roll
C Cut
D1 Diameter of the insertion member 2
D2 Diameter of the insertion member head 3
L Length of the insertion member
P Separation plane
T Thickness of the insertion member head 3

1. A piercing article having a longitudinal insertion member in the form of a pin, wherein, the piercing article is manufactured essentially in full of an alkylene-fluoralkylene copolymer material.

2. The piercing article according to claim 1, consisting mainly in full of an alkylene-fluoralky material formed without cutting.

3. The piercing article according to claim 1, wherein, the alkylene-fluoralkylene copolymer material is an ethylene tetrafluoioethylene (ETFE) material.

4. The piercing article according to claim 1, wherein the insertion member comprises an integrally formed insertion member head, with a diameter (D2) greater than a diameter (D1) of the insertion member.

5. The piercing article according to claim 1, wherein at least one locking member connectable with at least one free end of the insertion member.

6. The piercing article according to claim 5, wherein said locking member consists at least in part of an alkylene-fluoralkylene copolymer material, in particular an ETFE material.

7. The piercing article according to claim 5, wherein said locking member consists of a pharmaceutically compatible metal material.

8. The piercing article according to claim 5, wherein said locking member comprises connecting means with which the locking member is connectable with at least one free end of the insertion member.

9. The piercing article according to claim 6, wherein said alkylene-fluoralkylene copolymer material comprises a pharmaceutically compatible and/or food-safe dye.

10. The piercing article according to claim 1, wherein said alkylene-fluoralkylene copolymer material is provided with an anti-inflammatory agent (9).

11. An intermediary product for manufacturing a piercing article according to claim 1, comprising an elongated member in the form of a wire or rod of an alkylene-fluoralkylene copolymer material.

12. The intermediary product according to claim 11, wherein said alkylene-fluoralkylene copolymer material is an ethylene tetrafluoroethylene (ETFE) material.

13. The intermediary product according to claim 11, wherein said alkylene-fluoralkylene copolymer material comprises a pharmaceutically compatible and/or food-safe dye.

14. The intermediary product according claim 11, wherein said alkylene-fluoralkylene copolymer material is provided with an anti-inflammatory agent.

15. Use of an alkylene-fluoralkylene copolymer material for manufacturing the piercing article according to claim 1.
